(19) United States
(12) Patent Application Publication
(10) Pub. No.: US 2013/0078323 A1
(43) Pub. Date: Mar. 28, 2013

(54) ADAPTOGENIC ACTIVITY OF LABISIA PUMILA EXTRACT

(71) Applicants: Sarang Bani, Petaling Jaya (MY); Anjali Pandey, Petaling Jaya (MY); Kiranjeet Kour, Petaling Jaya (MY); Prashant Singh Chauhan, Petaling Jaya (MY); Balkrishan Chandan, Petaling Jaya (MY); Neelam Sharma, Petaling Jaya (MY); Kuldeep Singh, Petaling Jaya (MY); Surrinder Koul, Petaling Jaya (MY); Bishan Dutt Gupta, Petaling Jaya (MY); Payarelal Sangwan, Petaling Jaya (MY); Devinder Kumar Gupta, Petaling Jaya (MY); Shankar Lal, Petaling Jaya (MY); Rajendran Marnickavasagar, Petaling Jaya (MY)

(72) Inventors: Sarang Bani, Petaling Jaya (MY); Anjali Pandey, Petaling Jaya (MY); Kiranjeet Kour, Petaling Jaya (MY); Prashant Singh Chauhan, Petaling Jaya (MY); Balkrishan Chandan, Petaling Jaya (MY); Neelam Sharma, Petaling Jaya (MY); Kuldeep Singh, Petaling Jaya (MY); Surrinder Koul, Petaling Jaya (MY); Bishan Dutt Gupta, Petaling Jaya (MY); Payarelal Sangwan, Petaling Jaya (MY); Devinder Kumar Gupta, Petaling Jaya (MY); Shankar Lal, Petaling Jaya (MY); Rajendran Marnickavasagar, Petaling Jaya (MY)

(73) Assignee: HOLISTA BIOTECH SDN. BHD., Petaling Jaya, Selangor (MY)

(21) Appl. No.: 13/378,932

(22) PCT Filed: Jun. 16, 2009
(86) PCT No.: PCT/MY2009/000074

§ 371 Date: Jul. 3, 2012

(51) Int. Cl. A61K 36/185

(52) U.S. Cl. CPC A61K 36/185
USPC 424/774

(57) ABSTRACT

The present invention provides a process of extracting the leaves of Labisia pumila with water into a free flowing powder using accelerated solvent system and drying. The invention also relates to the immunopotentiating effects of the extract against stress. The extract has effects on fatigue, hypoxia time, potentiation of swimming endurance, bod weight, weight of the thymus and spleen and levels of CD244+ NK cells, CD4+ and CD8+ T cells, expression of ThI cytokines IL-2 and IFN-gamma, corticosterone, glutathione, alanine aminotransferase and lipid peroxidation levels.

### FIELD OF INVENTION

The present invention relates to adaptogenic activity of Labisia pumila extract.

### BACKGROUND ART

The adaptogen is a substance which can develop a state of raised resistance, [Lazarev NV. 7th All Union Congress Of Physiology, Biochemistry and Pharmacology 579. Medgiz, Moscow. Quoted from Brekhman, I. I., Dardymov, I. V., (1969). New Substances of plant origin which increase non-specific resistance. Annual Review of Pharmacology 1947; 9: 419-430.] enabling an organism to cope with stressful situations. The general aims of adaptogen therapy lie in its ability to reduce stress reactions during the alarm phase of the stress response, prevent or at least delay the state of exhaustion and, hence, provide a certain level of protection against long-term stress. A large variety of herbals have been studied for their adaptogenic and rejuvenating properties. These plants are believed to promote positive health and maintain organic resistance against infections by re-establishing body equilibrium and conditioning the body tissues [Bhagwandas, Fundamentals of Ayurvedic Medicine, Bansal Co., Delhi, India, ix-xvi (1978)).

Therapeutic approach for stress from ancient times has involved utilization of substances from natural origin, rather than synthesis of new chemical compounds. Since the introduction of adaptogens, several plants have been investigated, which were once used as tonics due to their adaptogenic and rejuvenating properties in traditional medicine [Rage N N, Thatte U M, Dahanukar S A. Adaptogenic properties of six rasayana herbs used in Ayurvedic medicine. Phytother Res 1999; 13:275-91].

Labisia pumila or Kacip Fatimah or “Akar Fatimah” [Burkill I H, A dictionary of the economic products of the Malay peninsula 1966, 2nd ed., vol. 1, A-H. Government of Malaysia and Singapore, Kuala Lumpur] as it is popularly known in Malaysia, is a plant of very high reputation in Malaysian traditional system of medicine. Traditionally, the water decoction of the root or the whole plant is consumed by Malaysian women for induction and facilitation of labour [Jamia A J and Houghton P J. Determination of iron content from Labisia pumila using inductively coupled plasma technique. Proceeding of the 16th National Seminar on Natural Products 2000; 118-120.].

Claimed traditional uses of the plant include use to treat dysentery, rheumatism and gonorrhoea. It is also used as anti-flatulence by helping to drive away and prevent the formation of gas. It has been claimed to be used as an anti-dysmenorrhoea [Rozihawati, Z., Aminah, H. Lokman, N., Preliminary trial on rooting ability of Labisia pumila cuttings, 2003. Malaysian Science & Technology Congress Agriculture Science, Cititel, Midvalley, Kuala Lumpur] helps in cleansing and avoiding painful or difficult menstruation.

### SUMMARY OF THE INVENTION

Accordingly, the present invention provides a process of extracting the leaves of Labisia pumila with water into a free flowing powder using accelerated solvent system and drying.

The present invention consists of several novel features and a combination of parts hereinafter fully described and illustrated in the accompanying description and drawings, it being understood that various changes in the details may be made without departing from the scope of the invention or sacrificing any of the advantages of the present invention.

### BRIEF DESCRIPTION OF THE DRAWINGS

The present invention will be fully understood from the detailed description given herein below and the accompanying drawings which are given by way of illustration only, and thus are not limitative of the present invention, wherein:

FIG. 1 is a bar graph representing the effect of KG, WS and Labisia pumila on CD4+ and CD8+ T cell population in chronically stressed mice;

FIG. 2 shows the effect of KG, WS and Labisia pumila on CD244+ population in chronically stressed mice;

FIG. 3 shows the effect of different doses of KG, WS and Labisia pumila on IFN gamma expression in chronically stressed mice;

FIG. 4 is a bar graph representing the comparative effect of KG, WS and Labisia pumila on IL-2 expression in chronically stressed mice;

FIG. 5 shows the effect of KG, WS and Labisia pumila on IL-4 production in stressed mice;

FIG. 6 shows the effect of KG, WS and Labisia pumila on plasma corticosterone levels in chronically stressed mice;

FIG. 7 shows the flow cytometric quadrant plot representation showing the comparative effect of KG, WS and Labisia pumila (effective doses only) on restraint stress induced depletion of T cell population;

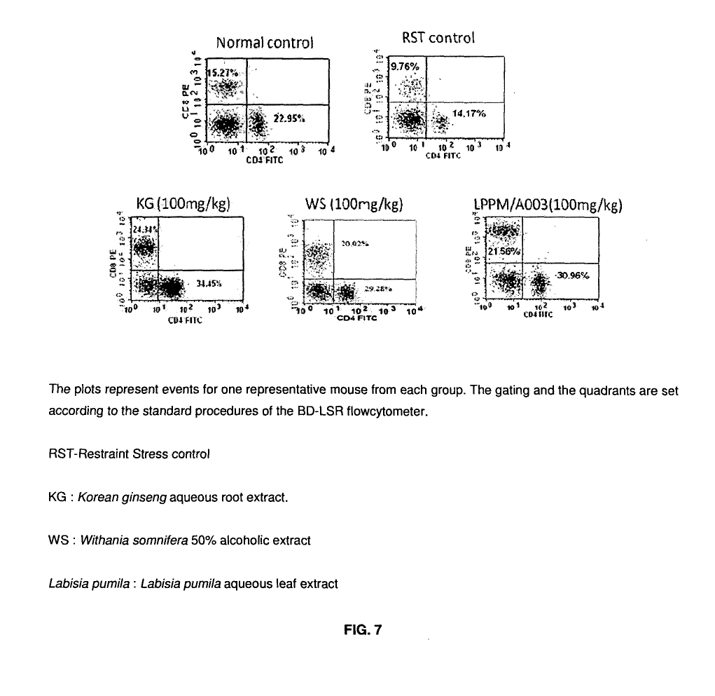

FIG. 8 shows the flow cytometric quadrant plot representation showing the comparative effect of KG, WS and Labisia pumila (effective doses only) on restraint stress induced depletion of NK cell population;

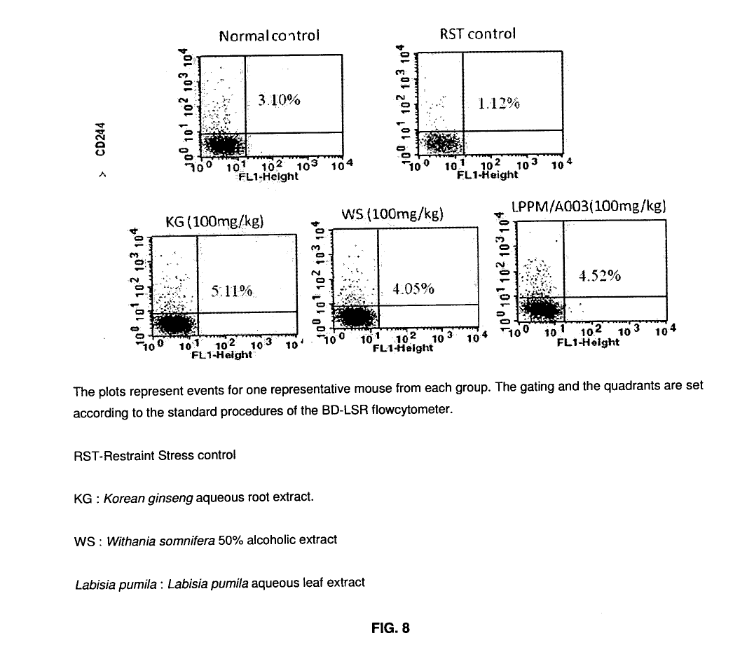

FIG. 9 shows the flow cytometric histogram representation showing the comparative effect of KG, WS and Labisia pumila (effective doses only) on IFN gamma expression;

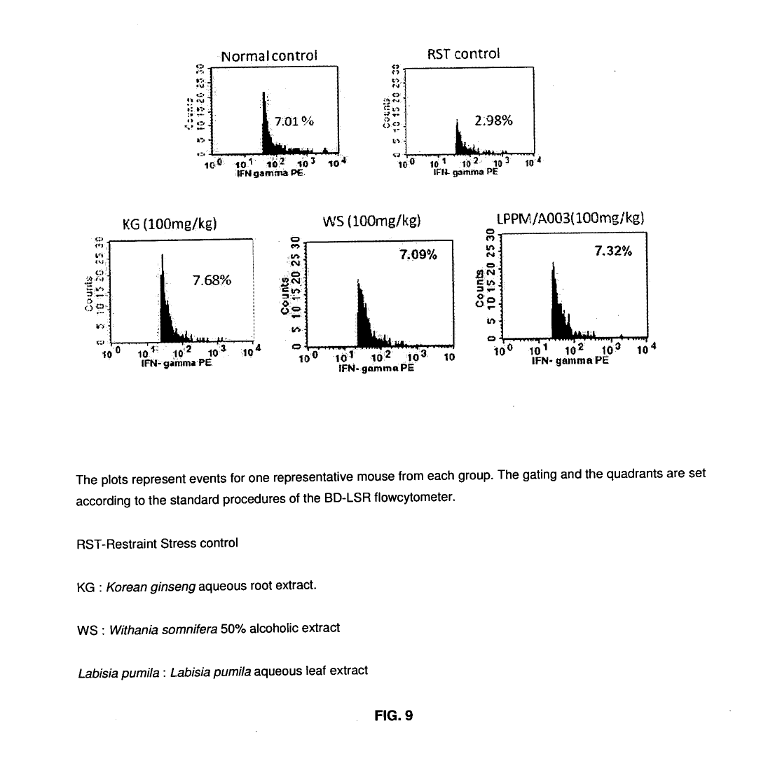

FIG. 10 shows the flow cytometric histogram representation showing the comparative effect of KG, WS and Labisia pumila (effective doses only) on IL-2 expression in stressed mice; and

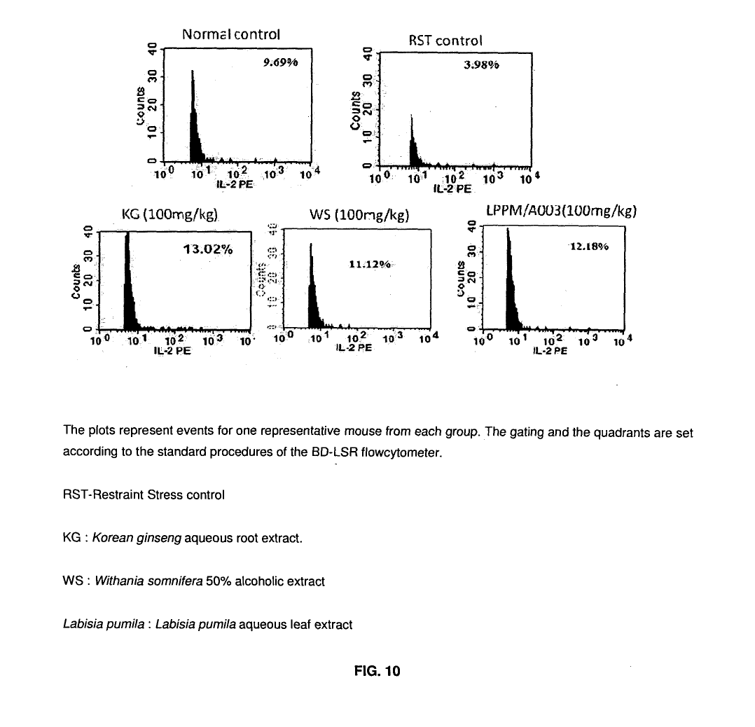

FIG. 11 show the flow cytometric histogram representation showing the comparative effect of KG, WS and Labisia pumila (effective doses only) on IL-4 expression on chronically stressed mice.

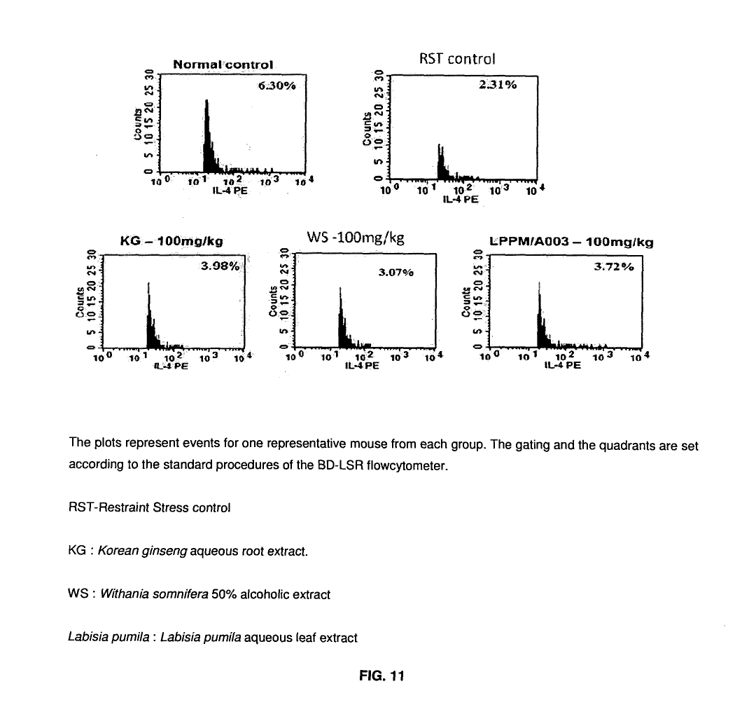

### DESCRIPTION OF THE PREFERRED EMBODIMENTS

The present invention relates to adaptogenic activity of Labisia pumila extract. Hereinafter, this specification will describe the present invention according to the preferred embodiments of the present invention. However, it is to be understood that limiting the description to the preferred embodiments of the invention is merely to facilitate discussion of the present invention and it is envisioned that those skilled in the art may devise various modifications and equivalents without departing from the scope of the appended claims.

The present invention relates to adaptogenic activity of Labisia pumila extract, obtained by extracting the leaves of Labisia pumila with water using accelerated solvent system and drying by known methods into a free flowing powder.

Labisia pumila aqueous leaf extract on submitting to pharmacological screening showed significant adaptogenic activity. The manifestation of stress for any reason causes deleterious changes and alters the normal functioning of the body, increases fatigue and induces mental depression. A true adaptogen is supposed to reverse such effects. The stress leads to central neuronal lesions since behavioural depression is a common consequence of stress and the antifatigue activity largely depends on neurochemicals, i.e. central NE and 5-HT. Our results show that pretreatment with Labisia pumila increased neuromuscular coordination and decreased fatigue. KG showed maximum effect at 100 mg/kg p.o. dose whereas WS showed most significant effect at 200 mg/kg p.o. In comparison Labisia pumila showed highly significant effect at 100 mg/kg p.o. dose and the effect was more than both KG and WS at all dose levels as illustrated in Table 1 below.

TABLE 1
Comparative effect of KG, WS and Labisia pumila on neuromuscular
coordination and fatigue in mice subjected to swimming induced stress.

| GROUPS | ANTIFATIGUE EFFECT (percent) | |
|---|---|---|
| (Dose mg/kg,p.o) | IMMEDIATE | AFTER 30 min |
| Normal control | 100 | 100 |
| (without stress) | | |
| Stress control | 0 | 25 |
| KG | 37.5 | 50* |
| (50) | | |
| KG | 62.5** | 75** |
| (100) | | |
| KG | 50* | 75** |
| (200) | | |
| WS | 12.5 | 37.5 |
| (50) | | |
| WS | 50* | 62.5* |
| (100) | | |
| WS | 62.5* | 62.5* |
| (200) | | |
| Labisia pumila | 37.5 | 37.5 |
| (50) | | |
| Labisia pumila | 62.5* | 87.5** |
| (100) | | |
| Labisia pumila | 37.5 | 87.5** |
| (200) | | |

The % effect of each group was determined by quantal response, i.e. all or nonresponse in a group.
(n = 8); **p < 0.001; *p < 0.01; Student's ‘t’ test
KG: Korean ginseng aqueous root extract.
WS: Withania somnifera 50% alcoholic extract
Labisia pumila: Labisia pumila aqueous leaf extract

The anti-hypoxia effect is related to improved or raised cerebral resistance to hypoxia and reduced cerebral consumption of oxygen in acute hypoxia. When mice are exposed to a hypobaric environment for a specified period, the mitochondria of heart and brain cells of mice are seriously damaged and brain neurotransmitters, i.e. norepinephrine (NE), dopamine (DA), serotonin (5-HT) and acetylcholine (ACh), are significantly decreased. Our results demonstrated that Labisia pumila in graded doses of 50-200 mg/kg, p.o. prolonged the hypoxia time in a dose-related manner (Table 2). The effect is probably related to an increase in the cerebral resistance to hypoxia and reducing the cerebral consumption of oxygen in acute hypoxia. The protective action of Labisia pumila on acute hypoxia mice may be due to the action of Labisia pumila on the pituitary-adrenal gland axis. Labisia pumila had 78.73% and 79.16% effect which was highly significant at the doses of 100 and 200 mg/kg p.o. respectively. In comparison KG showed maximum effect of 81.72% at 100 mg/kg oral dose and WS had 70.71% and 71.11% effect at oral doses of 100 and 200 mg/kg respectively as illustrated in Table 2 below.

TABLE 2
Effect of different doses of KG, WS and Labisia pumila on
acute hypoxiatime in normal mice.

| | | Hypoxia time | |
|---|---|---|---|
| | Dose | (in seconds) | % Activity against |
| Treatment | (mg/kg, p.o) | (Mean ± S.E) | normal control |
| Normal | — | 989.50 ±0.45 | — |
| Control | | | |
| KG | 50 | 1662.54 ± 0.28 | 68.011↑** |
| KG | 100 | 1798.13 ± 0.48 | 81.721↑** |
| KG | 200 | 1732.23 ± 0.36 | 75.061↑** |
| WS | 50 | 1512.34 ± 0.35 | 52.831↑* |
| WS | 100 | 1689.23 ± 0.42 | 70.711↑** |
| WS | 200 | 1693.15 ± 0.28 | 71.111↑** |
| Labisia | 50 | 1682.12 ± 0.56 | 69.991↑** |
| pumila | | | |
| Labisia | 100 | 1768.56 ± 0.48 | 78.731↑** |
| pumila | | | |
| Labisia | 200 | 1772.86 ± 0.38 | 79.161↑** |
| pumila | | | |

↑percent increase in the hypoxia time; Hypoxia Time expressed as the Mean ± S.E. (n = 8).
**p < 0.001; *p < 0.01 Student's ‘t’ test
KG: Korean ginseng aqueous root extract.
WS: Withania somnifera 50% alcoholic extract
Labisia pumila: Labisia pumila aqueous leaf extract

KG showed significant increase in swimming endurance time by 44.74% at the dose level of 200 mg/kg p.o. when compared to control group. WS showed maximum increase of 30.79% at 100 mg/kg p.o. dose level when compared to control. Labisia pumila showed highly significant effect of 63.74% increase at the dose level of 100 mg/kg p.o. as illustrated in Table 3 below. This showed Labisia pumila to have better endurance effect than both KG and WS in this model of swimming endurance test in experimental animals.

TABLE 3
Effect of KG, WS and Labisia pumila on swimming endurance in mice.

| | | Swimming endurance | % Activity |
|---|---|---|---|
| | Dose | (in minutes) | against |
| Treatment | (mg/kg, p.o) | (Mean ± S.E) | normal control |
| Normal | — | 147.25 ± 3.20 | — |
| Control | | | |
| KG | 50 | 192.23 ± 5.20 | 30.54↑* |
| KG | 100 | 201.25 ± 5.13 | 36.67↑* |
| KG | 200 | 213.13 ± 3.26 | 44.74↑** |
| WS | 50 | 178.13 ± 3.55 | 20.97↑ |
| WS | 100 | 192.6 ± 3.86 | 30.79↑* |
| WS | 200 | 189.32 ± 2.89 | 28.57↑ |
| Labisia | 50 | 214.72 ± 4.3 | 45.82↑* |
| pumila | | | |
| Labisia | 100 | 241.12 ± 4.3 | 63.741↑** |
| pumila | | | |
| Labisia | 200 | 239.3 ± 5.3 | 62.51↑** |
| pumila | | | |

↑: percent increase in swimming endurance; (n = 8).
**p < 0.001; *p < 0.01 Students ‘t’ test
KG: Korean ginseng aqueous root extract.
WS: Withania somnifera 50% alcoholic extract
Labisia pumila: Labisia pumila aqueous leaf extract

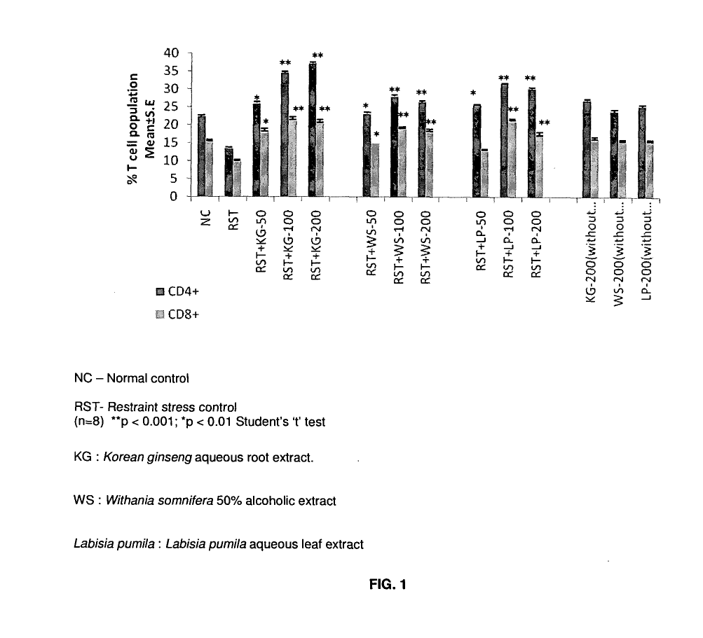

In response to stressful stimuli, the HPA axis activates resulting in enhanced release of glucocorticoid together with sympathetic mechanism which are mainly responsible for inhibition of cellular and humoral immune responses after psychological stress exposure. Flow cytometric analysis revealed that chronic restraint stress caused significant reduction in T lymphocytes population along with marked decrease in the expression of IL-2 (a growth factor for T cells), IFN-gamma (a signature cytokine for Th1 response) and IL-4, (a Th2 cytokine). The treatment of animals with Labisia pumila reversed the effect of restraint stress on these cell types and related cytokines showing it to have efficient antistress potential (as shown in accompanying FIGS. 1, 3, 4 and 5). The sympathetic nervous system in response to stress results in hyper secretion of corticosterone in case of rodents and cortisol in humans from the adrenal cortex and are the final effectors of organisms response to stressors. Labisia pumila decreased the chronic stress induced increase in plasma corticosterone levels having significant inhibition at 100 and 200 mg/kg p.o. dose levels and the effect was comparable to KG and WS which are known antistress agents, thereby, further confirming Labisia pumila to have stress busting potential.

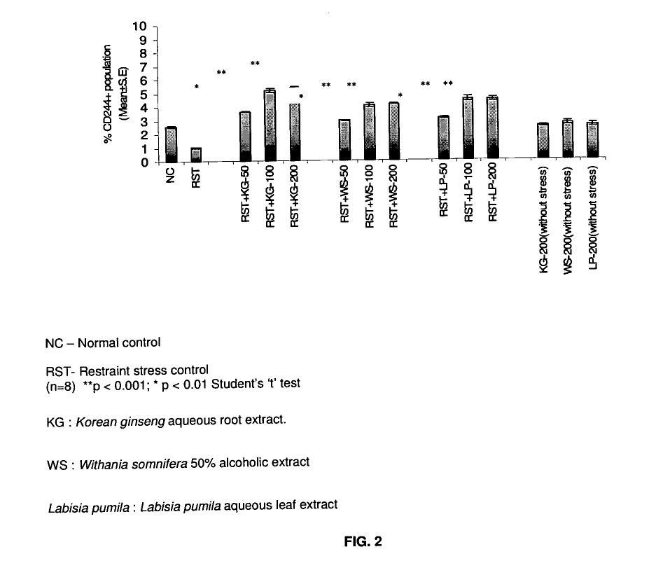

Natural killer cells (CD244+ cells) play a very important role in innate immune response that wards off the daily threat posed to the living being by the environment and the suppression of these cell types by stress makes the living being more prone to ailments. Labisia pumila recovered the stress induced depletion in the population of CD244+ natural killer cells (NK cells) showing significant effect at 100 and 200 mg/kg oral dose. WS showed maximum effect at 200 mg/kg p.o. dose but the effect was far less than Labisia pumila, KG, however, showed highly significant effect at 100 and 200 mg/kg p.o. as illustrated in FIG. 2.

During stress, nerve terminals accelerate the recruitment of lymphocytes to blood from spleen which is the major storage pool of lymphocytes. This results in squeezing of spleen causing reduction in weight as observed in chronic stress exposure. Prolonged activation of HPA axis results in increase in adrenal gland weight (hypertrophy). Also persistent high levels of coticosterone during chronic stress causes apoptosis and necrosis in immature T and B cells resulting in decline of thymus weight.

Stress of any kind results in a progressive deterioration in most of the endocrine functions and results in significant elevation in the activities of ALT as a result of an impairment of hepatic functions. GSH is the most important endogenous protective bio molecule against adverse conditions. In this connection the protective role of GSH against cellular lipid peroxidation has been well documented. A substantial increase in hepatic lipid peroxidation is evident from elevated MDA levels in liver homogenate with concurrent fall in hepatic GSH following physical and chemical stress is indicative that stress alters the physiological functioning of the body. The potential adaptogen combat adverse conditions and restored altered levels of the parameters to normal. The role of LPPM in attenuating these altered features may be visualized as a form of adaptation on the part of the GSH dependent defense system against lipid peroxidation. This unique effect of LPPM may be attributed to its anti oxidant properties which may inhibit the deleterious effect of free radicals generated by diverse stress situations. The treatment of animals with Labisia pumila significantly reversed the chronic stress induced adrenal hypertrophy and atrophy of spleen and thymus in a dose dependent manner with most significant effect at 100 mg/kg as illustrated in Table 5 below.

TABLE 5
Effect of KG, WS and Labisia pumila on Organ weights (mg) of
chronically stressed mice

| | Dose | | | ADRENAL |
|---|---|---|---|---|
| Treatment | mg/kg p.o. | THYMUS | SPLEEN | GLANDS |
| Naive control | — | 53.28 ± 1.2 | 62.0 ± 1.1 | 6.25 ± 0.81 |
| RST Control. | — | 38.15 ± 2.9 | 49.3 ± 1.9 | 11.75 ± 0.93 |
| KG-50 | 50 | 67.35 ± 2.5 | 69.65 ± 2.3 | 7 .98 ± 0.57 |
| | | (76.53↑)*^{a} | (41.27↑)*^{a} | (32.08)↓*^{a} |
| KG-100 | 100 | 69.65 ± 1.9 | 73.24 ± 1.65 | 7.13 ± 0.45 |
| | | (82.56↑)**^{a} | (48.55↑)**^{a} | (39.30)↓**^{a} |
| KG-200 | 200 | 62.75 ± 2.0 | 75.57 ± 2.0 | 7.01 ± 1.2 |
| | | (64.48↑)*^{a} | (53.28↑)**^{a} | (40.34)↓**^{a} |
| WS-50 | 50 | 59.12 ± 1.80 | 59.34 ± 2.1 | 9.67 ± 1.3 |
| | | (54.96↑)*^{a} | (20.36↑)*^{a} | (17.70)↓^{ns a} |
| WS-100 | 100 | 63.45 ± 2.4 | 65.29 ± 1.5 | 8.10 ± 2.5 |
| | | (66.31↑)**^{a} | (32.43↑)*^{a} | (31.06)↓*^{a} |
| WS-200 | 200 | 65.45 ± 2.7 | 66.45 ± 1.39 | 8.02 ± 1.0 |
| | | (71.55↑)**^{a} | (34.78↑)*^{a} | (31.74)↓*^{a} |
| Labisia pumila | 50 | 57.90 ± 2.1 | 58.45 ± 1.40 | 9.11 ± 0.8 |
| | | (51.76↑)*^{a} | (18.55↑)^{ns a} | (22.46)↓*^{a} |
| Labisia pumila | 100 | 65.45 ± 2.3 | 69.59 ± 2.6 | 7.73 ± 1.3 |
| | | (71.55↑)**^{a} | (41.15↑)**^{a} | (34.21)↓**^{a} |
| Labisia pumila | 200 | 67.70 ± 2.1 | 70.13 ± 3.0 | 7.22 ± 2.5 |
| | | (77.45↑)**^{a} | (42.25↑)**^{a} | (38.55)↓**^{a} |
| KG without | 200 | 56.28 ± 1.8 | 63.29 ± 1.7 | 6.57 ± 1.5 |
| stress | | (5.53↑)^{b} | (2.08↑)^{b} | (5.12)^{b} |
| WS without | 200 | 57.28 ± 2.1 | 63.29 ± 1.90 | 6.29 ± 0.57 |
| Stress | | (7.50↑)^{b} | (2.08↑)^{b} | (0.64)^{b} |
| Labisia pumila | 200 | 55.28 ± 1.90 | 62.24 ± 2.4 | 6.26 ± 1.9 |
| without stress | | (3.15↑)^{b} | (0.38↑)^{b} | (0.16)^{b} |

Values shown are Means ± SE for eight mice; Stress was induced was by immobilization RST.—Restraint stress control.
Values in parenthesis indicate the % activity of drug treated groups ^{a}against restraint stress control group. and ^{b}against naïve control.
Astericks indicate a statistically significant decrease/increase (*P < 0.01; **P < 0.001).
KG: Korean ginseng aqueous root extract.
WS: Withania somnifera 50% alcoholic extract
Labisia pumila: Labisia pumila aqueous leaf extract

It also normalized the chronic stress induced increase in serum alanine aminotransferase (ALT), alkaline phosphatase (ALP) and hepatic Lipid peroxidation (LP) levels and also augmented the CS induced decrease in hepatic Glutathione (GSH) as illustrated in Table 6 below. This shows the possible role of Labisia pumila in attenuating the activation of HPA axis. Labisia pumila showed significant effect at 100 and 200 mg/kg p.o. dose and it was more than WS at these dose levels. KG, however, showed better effect at 100 mg/kg oral dose as illustrated in Table 6 below.

TABLE 6
Effect on stress induced alteration of ALT, Lipid peroxidation (LP), and
Glutathione (GSH) in liver homogenate after 15 days treatment with
different doses of the Labisia pumila in comparison to WS, and KG.

| | Dose | ALT | | |
|---|---|---|---|---|
| | mg/ | μ mole | GSH | LP |
| | kg, | pyruvate/ | μ mole | μ mole MDA/g |
| Treatment | p.o. | min/It | GSH/gm | liv. |
| Vehicle | — | 61.31 ± 7.15 | 6.07 ± 0.29 | 95.37 ± 5.39 |
| control | | | | |
| Vehicle + | — | 83.52 ± 8.46^{b} | 3.49 ± 0.35^{b}*** | 133.06 ± 5.31^{b}*** |
| Stress | | | | |
| Labisia | 200 | 66.63 ± 7.01^{c} | 4.44 ± 0.30^{c} | 119.54 ± 3.65^{c} |
| pumila + | | (76.04)↓^{c} | (36.82) | (35.87) |
| Stress | | | | |
| LPPM/ | 100 | 71.55 ± 4.9^{c} | 4.14 ± 0.36^{c} | 123.06 ± 5.33^{c} |
| A003 + | | (53.89)↓^{c} | (25.19) | (26.53) |
| Stress | | | | |
| LPPM/ | 50 | 74.47 ± 7.33^{c} | 3.98 ± 0.22^{c} | 127.28 ± 4.98^{c} |
| A003 + | | (40.741)↓^{c} | (18.99) | (15.33) |
| Stress | | | | |
| WS + Stress | 200 | 65.70 ± 4.50^{c} | 4.90 ± 0.41^{c}* | 110.37 ± 5.49^{c}** |
| | | (80.23)↓^{c} | (54.65) | (60.20) |
| WS + Stress | 100 | 65.03 ± 2.99^{c} | 4.42 ± 0.16^{c} | 120.76 ± 5.48^{c} |
| | | (83.25)↓^{c} | (36.04) | (32.63) |
| WS + Stress | 50 | 69.42 ± 6.55^{c} | 4.07 ± 0.17^{c} | 125.76 ± 5.65^{c} |
| | | (63.48)↓^{c} | (22.48) | (19.36) |
| KG + Stress | 200 | 65.16 ± 3.60^{c} | 4.95 ± 0.40^{c}* | 108.45 ± 3.25^{c}** |
| | | (82.66)↓^{c} | (56.58) | (65.29) |
| KG + Stress | 100 | 70.75 ± 4.45^{c} | 4.47 ± 0.24^{c}* | 114.99 ± 5.87^{c}* |
| | | (57.49↓)^{c} | (37.98) | (47.94) |
| KG + Stress | 50 | 72.21 ± 3.95^{c} | 3.99 ± 0.09^{c} | 123.06 ± 6.79^{c} |
| | | (50.92)↓^{c} | (19.37) | (26.53) |
| Labisia | 200 | 61.57 ± 6.33^{b} | 5.96 ± 0.34^{b} | 94.22 ± 6.79^{b} |
| pumila | | (0.42)↑^{b} | (1.81) | (1.20) |
| without | | | | |
| Stress | | | | |
| WS | 200 | 58.10 ± 5.04^{b} | 5.99 ± 0.22^{b} | 96.91 ± 5.35^{b} |
| without | | (5.23)↓^{b} | (1.31) | (1.61) |
| Stress | | | | |
| KG without | 200 | 62.77 ± 3.57^{b} | 6.14 ± 0.26^{b} | 93.83 ± 4.99^{b} |
| Stress | | (6.57)↑^{b} | (0.48) | (1.61) |

Values represent mean ± SEM of eight animals in each group
^{b}Difference in relation to vehicle control mice
^{c}Difference in relation to stress control mice
*p < 0.05; **p < 0.01; ***p < 0.001 student's t Test.
KG: Korean ginseng aqueous root extract.
WS: Withania somnifera 50% alcoholic extract
Labisia pumila: Labisia pumila aqueous leaf extract

Process for preparation of Labisia pumila extracts is described below with the help of examples.

### Example 1

In one embodiment, 1 kg of Labisia pumila powdered dry leaves were extracted with millipore water (1:8) by heating at 80° C. for 3 hours, the contents drained off and the solid plant material recharged with equal volume of fresh solvent (water). The process was repeated three times more. All the combined extracts were then concentrated on rotavapor under reduced pressure at 50° C. temperature to give free flowing solid (99.98 g). The extract was standardized using HPLC on the basis of gallic acid used a marker compound. Column (RP-C₁₈, Merck, 5 μm, 4×250 mm), mobile phase containing solvent A (water containing 1% formic acid) and solvent B (acetonitrile) in gradient with % age of solvent B 5, 5, 55, 55, 5 & 5 at time interval 0, 5, 50, 55, 60 & 65 minutes respectively, flow rate 0.5 ml/min, temperature 30° C. and detection wavelength 270 nm run on LCMS-MS (Bruker Daltonics Esquire 3000). The HPLC chromatogram is shown in FIG. 1. The content of gallic acid was found 1.97% (w/w) in the extract.

### Example 2

In another, embodiment 1 kg of Labisia pumila dried leaves powder is extracted with double distilled water (1:8) by heating at 80° C. for 3 hours, the contents drained off and the solid plant material recharged with equal volume of fresh solvent (water). The process was repeated three times more. All the combined extracts were then lyophilized to give free flowing solid 94.96 gram. The extract was standardized in the same manner as shown in Example 1. Gallic acid content was found 2.02% in the extract (w/w).

### Example 3

In yet another embodiment, the aqueous extract of Labisia pumila was prepared by taking three thimbles each comprising dry powdered leaves (20 g) with distilled water (1:8) using accelerated solvent extraction with temperature 40° C. for 15 minutes, the contents drained off and the solid plant material recharged with equal volume of fresh solvent (water). The process was repeated three times more. All the combined extracts were then concentrated on rotavapor under reduced pressure at 50° C. temperature to give free flowing solid 4.85 gram. The extract was standardized in the same conditions as shown in Example 1 and gallic acid content was found to be 1.81% in the extract (w/w).

### Example 4

In yet another embodiment, the aqueous extract of Labisia pumila was prepared by taking three thimbles each comprising dry powdered leaves (20 g) with distilled water (1:8) using accelerated solvent extraction with temperature 60° C. for 15 minutes, the contents drained off and the solid plant material recharged with equal volume of fresh solvent (water). The process was repeated three times more. All the combined extracts were then concentrated on rotavapor under reduced pressure at 50° C. temperature to give free flowing solid 6.02 g. The extract was standardized in the same conditions as shown in Example 1 and gallic acid content was found to be 2.01% in the extract.

### 1. Animals:

The pharmacological studies were conducted on Swiss albino mice (25-30 g) of either sex, colony bred in the Institute's animal house. After procurement, all the animals were divided into different groups and left for one week for acclimatization to experimentation room and maintained in standard conditions (23±2° C. experimental room temperature, 60-70% relative humidity and 12 h, photo period). The animals were fed with standard rodents pellet diet and were provided water ad libitum.

### 2. Anti-Fatigue Effect.

Labisia pumila, KG and WS at dose levels of 50, 100 and 200 mg/kg were administered to the test groups. One normal control group (without swimming stress) and other swimming stress control group were used for the study.

Pre-trained Swiss albino mice which stayed on a rotating rod (UGO Basile, Italy) at 20 rpm, for more than 5 minutes in three successive trials for 5 consecutive days, were used in this study. Drug administration was carried out for 14 days. Animals were given three successive trials to stay on rotating rod regularly during the course of study. On day 15, one hour after treatment, animals of all the groups except group-1 i.e. control without stress (Table 1) were exhausted by swimming continuously for two hours. The animals were immediately taken out, dried with tissue paper and placed on the rotating rod to monitor anti-fatigue and motor coordination effects. The number of mice that stayed on the rota-rod for 180 seconds or more were considered as untired with motor coordination. The percent effect of each group was calculated on the basis of the number of mice that stayed on the rota-rod for >180 seconds (by all or non-method). The same animals were again placed on the rota-rod after 30 minutes of removal from the swimming bath, to monitor the anti fatigue effect once again.

Similarly, the animals of control group (control without stress) which were not allowed to swim were also placed on the rotating rod for to see the anti fatigue effect in normal animals.KG showed maximum effect at 100 mg/kg p.o. dose whereas WS showed most significant effect at 200 mg/kg p.o. In comparison Labisia pumila showed highly significant effect at 100 mg/kg p.o. dose and the effect was more than both KG and WS at all dose levels (Table 1).

### 3. Hypoxia Test

Labisia pumila, KG and WS were administered at the dose level 50, 100 and 200 mg/kg continuously for 14 days to the swiss albino mice. On day 15, one hour after treatment, the hypoxia time was recorded individually or each animal by placing the animal in an empty glass jar of 300-ml capacity attached to an electronic watch. The jars were made air-tight with greased glass stoppers and the time until onset of convulsion was recorded as the end point. Labisia pumila showed 78.73% and 79.16% increase in time when compared to control at 100 and 200 mg/kg p.o. dose levels respectively. KG showed the most significant effect at 100 mg/kg p.o. dose where it showed 81.72% increase against normal control. WS at 100 and 200 mg/kg p.o. dose levels showed 70.71% and 71.11% increase in time when compared to control (Table 2). Thus Labisia pumila showed highly significant effect that was more than WS and was close to the effect shown by KG.

### 4. Swimming Endurance Test:

Male Swiss albino mice were divided into six groups (n=6) in which one group served as normal control and the animals treated with Labisia pumila, KG and WS at 50, 100 and 200 mg/kg served as the drug treated group. Test material was administered orally once a day for 14 days. On day 15, one hour after drug administration, the swimming time of each animal was measured individually. The animals were allowed to swim inside a perplex glass beaker (30 cm high with 20 cm diameter, containing water up to 25 cm height) maintained at 26±1° C. The mice were allowed to swim till they got exhausted was considered as the endpoint. The mean swimming time for each group was calculated. Labisia pumila showed highly significant effect of 63.74% increase at the dose level of 100 mg/kg p.o. KG showed significant increase in swimming endurance time by 44.74% at the dose level of 200 mg/kg p.o. when compared to control group and WS showed maximum increase of 30.79% at 100 mg/kg p.o. dose level when compared to control. (Table 3). This shows Labisia pumila to be more effective as a physical endurance enhancer than both KG and WS as it had most significant increase in the swimming endurance time in experimental animals.

### 5. Chronic Restraint Stress:

Male Swiss albino mice, 10-12 weeks old and weighing about 20-22 grams were employed for this study. Mice were restrained in these 50 ml conical polypropylene tubes for 12 h during the dark cycle (2000-0800 h) for 14 days. Experimental animals were divided into groups of eight animals each. Group-1 served as naive control group without any restraint stress conditions. Group-2 was subjected to restraint stress and served as restraint stress control (RST-control). Group 3-11 were the drug treated groups that received Labisia pumila, KG and WS at oral doses of 50, 100 and 200 mg/kg respectively. Group 12, 13 and 14 were the treated groups in which animals were not subjected to restraint stress and all the test samples were administered daily for the duration of experiment i.e. for 14 days at the dose levels of 200 mg/kg p.o dose.

### 5.1. Lymphocyte Immunophenotyping.

On day 14, blood was taken from the retro-orbital plexus of animals from all the groups for the assessment of various immune cells surface markers. Murine monoclonal antibodies conjugated to a fluorochrome and directed against co-receptors CD⁴+ T helper cells, CD8⁺ T cytotoxic cells and CD244⁺ Natural killer cells were used in a multi parametric flowcytometric assay to quantify the lymphocyte subsets associated with the cell-mediated immune response. These flourochrome labeled monoclonal antibodies were added directly to 100 μl of whole blood, which was then lysed using whole blood lysing reagent (BD Biosciences). Following the final centrifugation, samples were resuspended in phosphate buffer saline (pH, 7.4) and analyzed directly on the flowcytometer (BD Biosciences) using Cell Quest Pro Software (BD Biosciences). Labisia pumila showed highly significant recovery in the restraint stress induced depleted CD4+ and CD8+ T cell population. KG however showed more effect than Labisia pumila at the same dose levels i.e. 100 and 200 mg/kg. WS showed less effect than Labisia pumila (FIG. 1). Natural killer cells (NK) are important component of innate immune response and are adversely effected by stress. Labisia pumila showed significant recovery of CD244+ NK cells at 100 and 200 mg/kg dose levels and the effect was comparable to WS. KG, however, showed better effect than both WS and Labisia pumila (FIG. 2).

### 5.2. Intracellular Cytokine Estimation

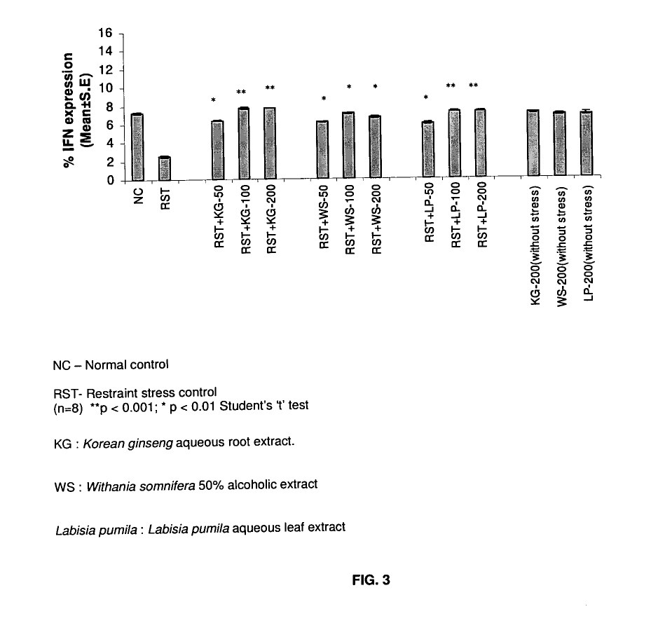

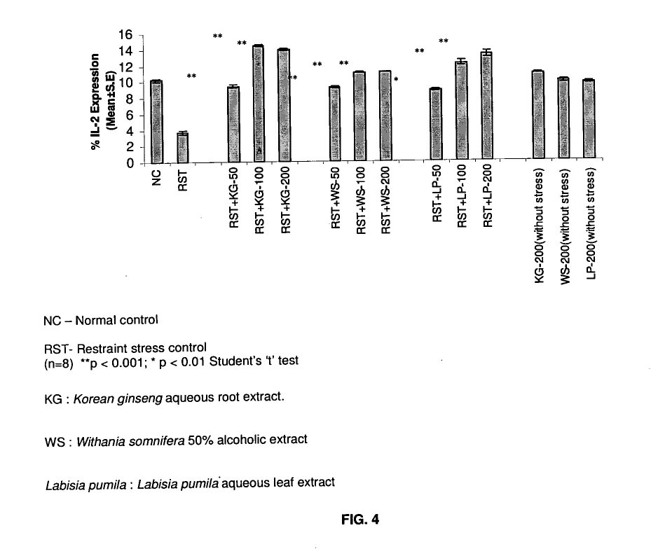

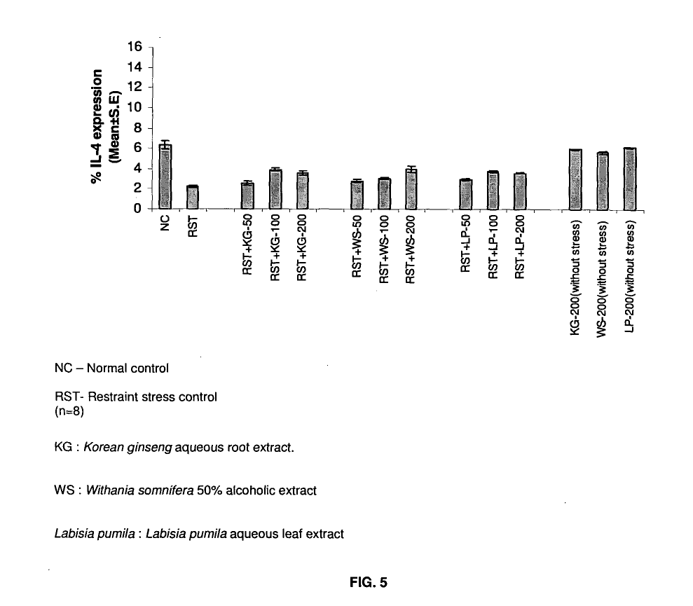

100 μl of the whole blood was taken in the falcon tubes. Phycoerythrin (PE) labeled IFN-γ monoclonal antibodies were used in one set, PE labeled IL-2 monoclonal antibody was used in the second set and PE labeled IL-4 monoclonal antibody was used in third set of experimentation. The tubes were incubated in dark for 30 min at room temperature. 2 ml of 1×FACS lysing solution (BD Biosciences) was then added with gentle mixing followed by incubation for 10 min. at room temperature. The samples were centrifuged (300-400 g) for 10 min. The supernatant was aspirated and the sample was resuspended in 500 μl of PBS. Acquisition and the analysis were done directly on flowcytometer using Cell Quest Pro software (BD Biosciences). Chronic restrained stress condition suppressed the expression of IFN-γ and IL-2 to 2.98% and 3.98% when compared to normal control expressing 7.01% IFN-γ and 9.69% IL-2 respectively. Labisia pumila showed significant recovery in the expression of IFN-γ and IL-2 which was 7.32% and 12.18% when compared to stress control (as shown in FIGS. 3 and 4). However, it did not show significant increase in the expression of IL-4 (FIG. 5) thereby, showing a Th1 specific response as IFN-γ is a specific Th1 marker cytokine.

### 5.3. Corticosterone Assay.

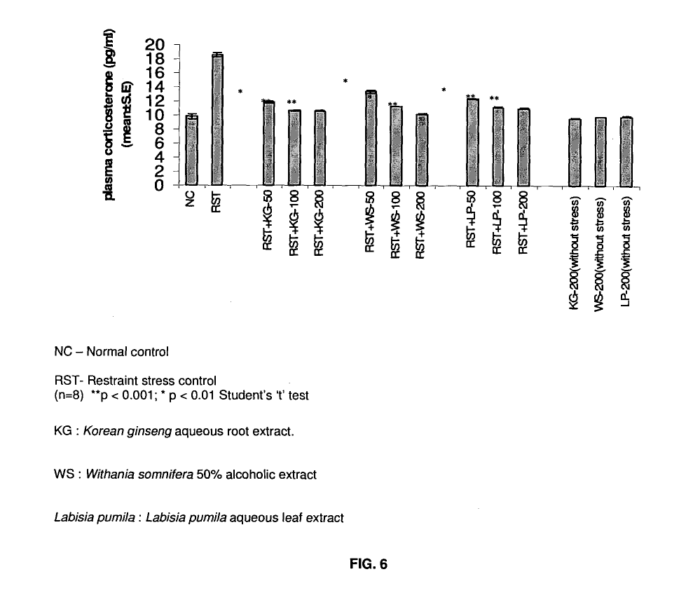

Corticosterone in rats and cortisol in humans are the final effectors of HPA axis activation during stress exposure. Coticosterone was assessed in animals subjected to restraint stress to observe the effect of drug on plasma corticosterone levels. On day 14, blood was collected from all the groups. The plasma was separated and corticosterone levels were assessed by competitive immune-enzymatic method (Elisa kit Neogen Corporation). All samples were assayed in triplicates at a wavelength of 450 nm. Labisia pumila reduced the increased levels of plasma corticosterone levels and the effect was highly significant at 100 mg/kg dose level (FIG. 6). KG and WS also had significant inhibitory effect with KG showing significant effect at 200 mg/kg p.o. dose (FIG. 6).

### 5.4. Body and Organ Weights.

After the last stress session, the body weights of all the animals from all the groups were taken following which the animals were sacrificed and their thymus, adrenal glands, and spleen, were removed and weighed. KG showed increase in body weight of the animal by 4.67% at the dose level of 100 mg/kg p.o. when compared to the depleted body weight of restrained stress control group. WS showed maximum increase of 2.50% at 100 mg/kg p.o. dose level when compared to control whereas Labisia pumila also showed 2.50% increase at the dose level of 100 mg/kg p.o. as illustrated in Table 4 below.

TABLE 4
Effect of KG, WS and Labisia pumila on body weights in mice.

| | | | | Change in |
|---|---|---|---|---|
| | Dose | Body weights (g) | | Body Wt. |
| Treatment | mg/kg p.o. | DAY 0 | DAY 14 | (g) |
| Naive | — | 22.83 ± 0.30 | 24.66 ± 0.21 | 1.83↑ |
| control | | | | |
| RST | — | 22.50 ± 0.22 | 17.16 ± 0.30 | 5.34↓ |
| Control. | | | | |
| KG | 50 | 23.16 ± 0.22 | 26.0 ± 0.36 | 2.84↑ |
| KG | 100 | 22.66 ± 0.33 | 27.33 ± 0.55 | 4.67↑ |
| KG | 200 | 22.83 ± 0.30 | 28.50 ± 0.22 | 5.67↑ |
| WS | 50 | 22.16 ± 0.16 | 24.50 ± 0.22 | 2.35↑ |
| WS | 100 | 22.50 ± 0.22 | 25.0 ± 0.51 | 2.50↑ |
| WS | 200 | 22.16 ± 0.30 | 24.83 ± 0.54 | 2.67↑ |
| Labisia | 50 | 22.50 ± 0.42 | 23.83 ± 0.30 | 1.33↑ |
| pumila | | | | |
| Labisia | 100 | 22.0 ± 0.23 | 24.5 ± 0.22 | 2.50↑ |
| pumila | | | | |
| Labisia | 200 | 22.16 ± 0.16 | 24.50 ± 0.22 | 2.34↑ |
| pumila | | | | |
| KG without | 200 | 23.0 ± 0.25 | 28.12 ± 0.23 | 5.12↑ |
| stress | | | | |
| WS without | 200 | 23.12 ± 0.23 | 25.65 ± 0.26 | 2.53↑ |
| Stress | | | | |
| Labisia | 200 | 23.65 ± 0.33 | 25.95 ± 0.24 | 2.30↑ |
| pumila | | | | |
| without | | | | |
| stress | | | | |

Values shown are Means ± SE for eight mice;
RST.—Restraint stress control;
KG: Korean ginseng aqueous root extract.
WS: Withania somnifera 50% alcoholic extract
Labisia pumila: Labisia pumila aqueous leaf extract

KG, WS and Labisia pumila showed highly significant increase in the weights of thymus and spleen, showing enhanced activity of 82.56% (thymus), 48.55% (spleen) in KG at 100 mg/kg dose; increase of 66.31% (thymus), 32.43% (spleen) in WS at 100 mg/kg and significant increase of 71.55% (thymus), 41.15% (spleen) in Labisia pumila at 100 mg/kg dose respectively. The hypertrophy of adrenal gland was significantly reduced by 34.21% in animals treated with Labisia pumila at 100 mg/kg dose compared to stressed control (Table 5).

### 5.5 Evaluation of Biochemical Parameters.

Before sacrificing the animal, Blood was collected from the orbital sinus from all the animals, serum separated for different estimations:(ALT) alanine aminotransferase. After sacrificing these animals, their livers were quickly excised, cleaned of adhering tissue, weighed and homogenized in chilled phosphate buffer saline for the analysis of lipid peroxidation. Labisia pumila showed significant restorative and curative activity and the effect was highly significant and dose related. There was dose related ALT reduction in the stress induced elevated levels of ALT and the effect were comparable to KG and WS. GSH levels were increased in the animals that were administered Labisia pumila in graded doses. LP levels were also normalised as stress raises LP levels and dose related reduction in its level was observed in Labisia pumila treated animals. (Table 6).

The present invention therefore provides that Labisia pumila exhibits adaptogenic potential that is more significant than WS and is comparable to KG and will be useful to underline importance of systems approaches in the ethnopharmacology based drug discovery. Such plant extracts may deliver appropriate synergistic moieties, which concurrently or simultaneously modulate and restores homeostatic conditions.

It has identified in the present application that Labisia pumila is a potent adaptogen with broad spectrum of activities, that Labisia pumila scores over KG and WS extracts in physical endurance study in mice, that Labisia pumila shows better anti fatigue potential than KG and WS extracts by showing better muscular coordination in fatigued mice, that Labisia pumila, KG and WS extracts had comparable anti-hypoxia potential in mice, that Labisia pumila showed increase in stress induced depleted CD4 and CD8 positive T cell population and the effect was more than WS at the oral doses of 100 and 200 mg/kg in chronically stressed mice, that stressed animals treated with Labisia pumila showed greater recovery of IL-2 & IFN-gamma than WS, thereby, showing better antistress potential with essentially a Th1 response against WS, that Labisia pumila showed dose related suppression of stress induced release of corticosterone and the effect was more than WS and was comparable to KG, that Labisia pumila could be a suitable candidate with its adaptogenic effects providing the basis for extending its use as an physical endurance enhancer, anti fatigue, anti stress and as immunostatic agent and that Labisia pumila could be a candidate as an adaptogen in combination therapy.

1. A process of extracting the leaves of Labisia pumila with water into a free flowing powder using accelerated solvent system and drying.

2. The process as claimed in claim 1, wherein the Labisia pumila aqueous leaf extract is administered systemically and orally.

3. The process as claimed in claim 1, wherein adaptogen activity of the Labisia pumila aqueous leaf extract provides anti-fatigue effect with maximum effect at 100 mg/kg p.o.

4. The process as claimed in claim 1, wherein adaptogen activity of the Labisia pumila aqueous leaf extract provides enhancement in the Hypoxia time.

5. The process as claimed in claim 1, wherein adaptogen activity of the Labisia pumila aqueous leaf extract provides higher potentiation in swimming endurance.

6. The process as claimed in claim 1, wherein adaptogen activity of the Labisia pumila aqueous leaf extract provides recovery of restraint stress induced depletion of CD244+ NK cells.

7. The process as claimed in claim 6, wherein adaptogen activity of the Labisia pumila aqueous leaf extract provides recovery in the restraint stress induced depleted CD4+ and CD8+ T cell population.

8. The process as claimed in claim 7, wherein adaptogen activity of the Labisia pumila aqueous leaf extract provides increased expression of Th1 cytokines IL-2 and IFN-gamma in restraint stress induced experimental animals.

9. The process as claimed in claim 1, wherein adaptogen activity of the Labisia pumila aqueous leaf extract reduces the stress induced increased levels of plasma corticosterone and the effect was highly significant at 100 mg/kg dose level.

10. The process as claimed in claim 1, wherein adaptogen activity of the Labisia pumila aqueous leaf extract shows maximum increase of 2.50% in body weight at 100 mg/kg p.o. dose level when compared to the decreased body weight of restrained stress control group.

11. The process as claimed in claim 1, wherein adaptogen activity of the Labisia pumila aqueous leaf extract shows increase in the weights of thymus and spleen.

12. The process as claimed in claim 1, wherein adaptogen activity of the Labisia pumila aqueous leaf extract shows dose related significant inhibition in the stress induced elevated levels of alanine aminotransferase (ALT).

13. The process as claimed in claim 1, wherein adaptogen activity of the Labisia pumila aqueous leaf extract shows dose related significant recovery (increase) in the stress induced depleted levels of Glutathione (GSH).

14. The process as claimed in claim 1, wherein adaptogen activity of the Labisia pumila aqueous leaf extract shows dose related significant reduction (decrease) in the stress induced increased Lipid peroxidation (LP) levels.
